(19) United States
(12) Patent Application Publication
(10) Pub. No.: US 2025/0154082 A1
(43) Pub. Date: May 15, 2025

(54) CONTAMINANT REMOVAL AND REDUCTION SYSTEM

(71) Applicant: Honeywell International Inc., Charlotte, NC (US)

(72) Inventors: Phoebe Henson, Glendale, AZ (US); Jack Ford, Phoenix, AZ (US); Meghan Pipitone, Scottsdale, AZ (US); Nathaniel Gressly, Phoenix, AZ (US); Rebecca Kamire, Buffalo Grove, IL (US); Emir Rahislic, Chicago, IL (US); Mark Triezenberg, Westmont, IL (US)

(21) Appl. No.: 18/505,531

(22) Filed: Nov. 9, 2023

(51) Int. Cl. C07C 7/11; B01D 53/14; B01D 53/26; C07C 1/12; C07C 7/00

(52) U.S. Cl. CPC C07C 7/11; B01D 53/1425; B01D 53/1475; B01D 53/268; C07C 1/12; C07C 7/005; B01D 2257/504; B01D 2257/80

(57) ABSTRACT

A system for generating methane includes a scrubber, a stripper, a vacuum pump, a water separator, and a Sabatier reactor. The scrubber is configured to absorb one or more contaminants from an air stream into a liquid sorbent, in which the one or more contaminants include carbon dioxide. The stripper is configured to desorb the one or more contaminants from the liquid sorbent. The vacuum pump is configured to pressurize the desorbed contaminants to a reaction pressure of the carbon dioxide. The water separator is configured to remove water from the pressurized, desorbed contaminants. The Sabatier reactor is configured to generate the methane from the carbon dioxide of the pressurized, desorbed contaminants.

### GOVERNMENT RIGHTS

This invention was made with Government support under Grant Contract Number 80MSFC18C0045 awarded by National Aeronautics and Space Administration (NASA) Marshall Space Flight Center. The Government has certain rights in the invention.

### TECHNICAL FIELD

The present disclosure relates to systems and techniques for generating methane from contaminants.

### BACKGROUND

An environmental control system (ECS) of a structure, such as a building or vehicle, may remove carbon dioxide expelled by occupants of an environment, such as a room or cabin, to maintain comfort and safety. In some instances, the carbon dioxide may be absorbed from the environment by a liquid sorbent and desorbed from the liquid sorbent for discharge from the structure. However, for an atmosphere limited structure, such as a spacecraft or submarine, such discharge of carbon dioxide may waste oxygen from the carbon dioxide that may otherwise be recovered. To extract oxygen from the carbon dioxide, the ECS may capture and store the carbon dioxide in pressurized vessels for discharge or further use.

### SUMMARY

The disclosure describes systems and techniques for removing contaminants, such as carbon dioxide, using a liquid sorbent and directly reducing the contaminants, such as by generating methane from removed carbon dioxide. The contaminants are absorbed into the liquid sorbent at a scrubber and desorbed from the liquid sorbent at a stripper. Rather than discharge the contaminants to an atmosphere or store the contaminants in pressurized vessels for future processing, carbon dioxide, and optionally other contaminants, may be directly fed into a Sabatier reactor to be reacted and/or reduced. For example, carbon dioxide may react with hydrogen in the Sabatier reactor to produce methane. By directly incorporating contaminant removal and contaminant processing functions, systems described herein may have lower size, weight, power, and complexity compared to systems that store contaminants for future discharge or processing. For example, these systems may avoid large storage vessels, recycle heat between relatively hot product streams and relatively cool liquid sorbent streams, and/or pressurize the carbon dioxide to a lower pressure, among other advantages that may result from direct incorporation of the contaminant removal and processing functions.

In some examples, the disclosure describes a system for generating methane. The system includes a scrubber, a stripper, a vacuum pump, a water separator, and a Sabatier reactor. The scrubber is configured to absorb one or more contaminants from an air stream into a liquid sorbent, in which the one or more contaminants include carbon dioxide. The stripper is configured to desorb the one or more contaminants from the liquid sorbent. The vacuum pump is configured to pressurize the desorbed contaminants to a reaction pressure of the carbon dioxide. The water separator is configured to remove water from the pressurized, desorbed contaminants. The Sabatier reactor is configured to generate the methane from the carbon dioxide of the pressurized, desorbed contaminants.

In some examples, the disclosure describes a method for generating methane. The method includes absorbing, by a scrubber, one or more contaminants from an air stream into a liquid sorbent, including carbon dioxide, and desorbing, by a stripper, the one or more contaminants from the liquid sorbent. The method further includes conditioning the desorbed contaminants by pressurizing, by a vacuum pump, the desorbed contaminants to a reaction pressure of the carbon dioxide and removing, by a water separator, water from the pressurized, desorbed contaminants. The method further includes generating, by a Sabatier reactor, the methane from the carbon dioxide of the pressurized, desorbed contaminants.

### BRIEF DESCRIPTION OF THE FIGURES

The details of one or more examples are set forth in the accompanying drawings and the description below. Other features, objects, and advantages will be apparent from the description and drawings, and from the claims.

FIG. 1A is a block diagram illustrating an example contaminant processing system for producing oxygen and carbon from carbon dioxide.

FIG. 1B is a block diagram illustrating an example methane generation system for generating methane from carbon dioxide.

FIG. 1C is a schematic diagram illustrating an example contaminant processing system that includes a methane generation system for generating methane from carbon dioxide.

FIG. 2 is a schematic diagram illustrating an example Sabatier assembly of a methane generation system.

FIG. 3 is an example flowchart of a method for generating methane from carbon dioxide.

FIG. 4 is a block diagram illustrating an example contaminant processing system for producing oxygen and hydrocarbons from carbon dioxide.

FIG. 5 is a block diagram illustrating an example carbon generation system for generating carbon from carbon dioxide.

### DETAILED DESCRIPTION

The disclosure describes systems and techniques for removing contaminants, such as carbon dioxide, from an air stream using liquid sorbents and reducing the contaminants, such as by generating methane from removed carbon dioxide. Contaminant reduction systems described herein may be utilized as part of an environmental control system (ECS), such as in spacecraft, aircraft, watercraft, and the like. In some examples, contaminant reduction systems may be used in an ECS of a resource-limited environment, such as a spacecraft. Such resource-limited environments may be particularly suited for a contaminant reduction system that includes components that use low amounts of power and operate with lower complexity.

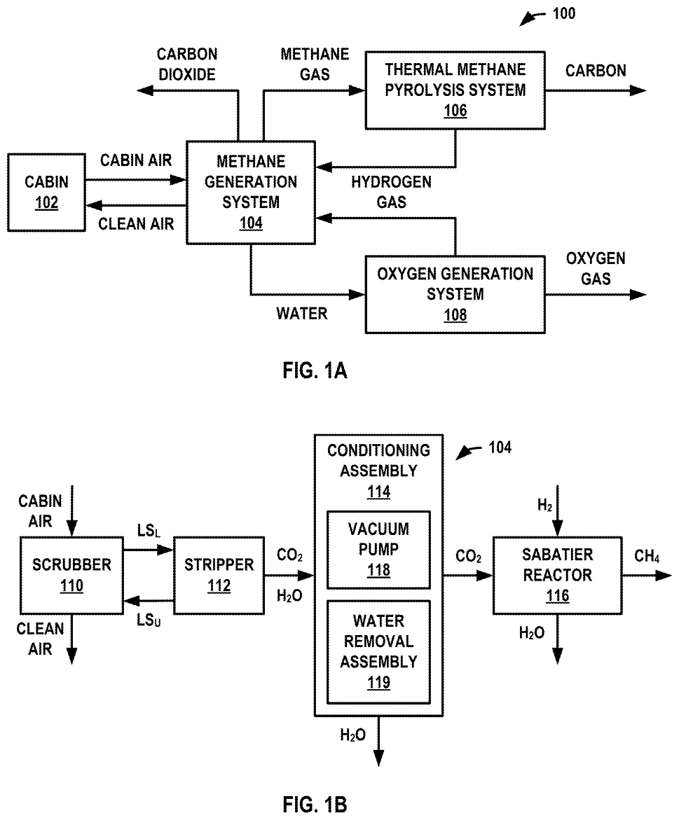

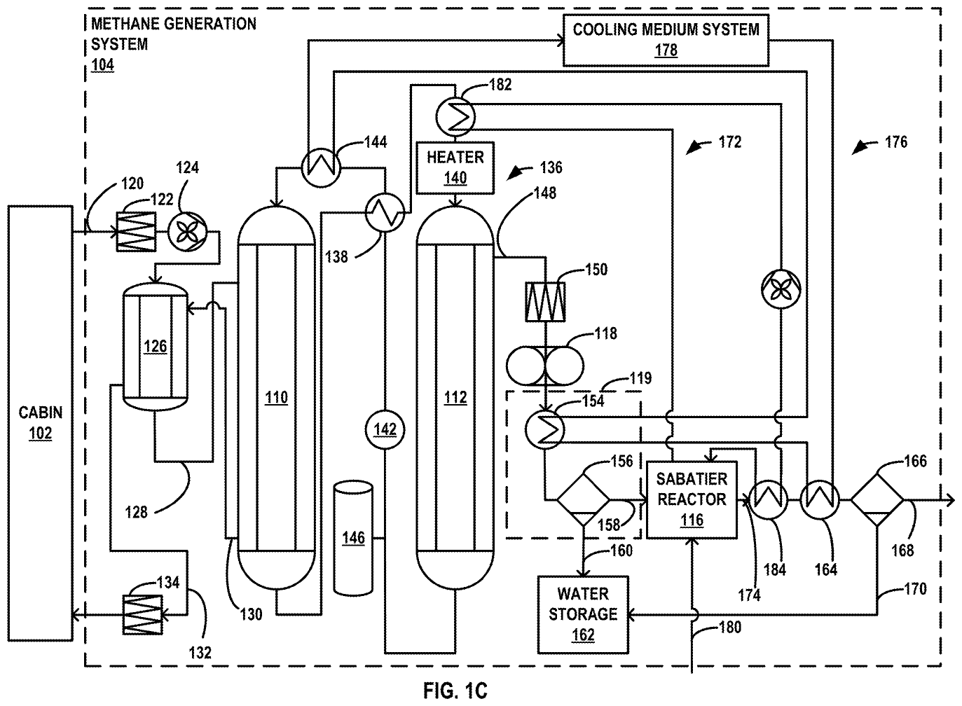

FIG. 1A is a block diagram illustrating an example contaminant removal and processing system 100 for producing oxygen and carbon from carbon dioxide removed from a cabin 102. Cabin 102 may include any enclosed and controlled atmosphere, and may include any combination of personnel, machinery, or equipment. Cabin 102 may be a controlled environment, such as an aircraft cabin, spacecraft cabin, watercraft cabin, or the like, and contaminants removed from cabin 102 may include, but are not limited to, carbon dioxide, water, hydrocarbons, permanent gases, or the like. In some examples, cabin 102 is a cabin of a closed-loop system, such as a spacecraft cabin or submarine cabin, in which components of a cabin air stream from cabin 102, such as carbon dioxide and water, may be removed within system 100, allowing a purified supply air stream to be generated and carbon dioxide and water to be recovered. However, in other examples, cabin 102 is a cabin of an open-loop system, such as an aircraft cabin, in which contaminants of a cabin air stream may be removed to generate a purified supply air stream with only partial recovery of the contaminants.

System 100 includes a methane generation system 104 as a contaminant reduction system, a methane pyrolysis system 106, and an electrolysis system 108. Methane generation system 104 is configured to both remove contaminants from an air stream and generate methane from the removed contaminants. To remove the contaminants, methane generation system 104 is configured to absorb one or more contaminants from the air stream, such as a cabin air stream, into a liquid sorbent and desorb the one or more contaminants from the liquid sorbent. The one or more contaminants include carbon dioxide, such that a clean air stream discharged from methane generation system 104 has a lower concentration of carbon dioxide than the cabin air stream received by methane generation system 104.

Methane generation system 104 reacts hydrogen with the carbon dioxide through an exothermic Sabatier reaction to generate methane and water. Methane produced by methane generation system 104 may be used for a variety of purposes. In the example of FIG. 1A, the methane is used to generate oxygen and solid carbon via generation of hydrogen for methane generation system 104. Methane pyrolysis system 106 is configured to generate hydrogen from pyrolysis of the methane. Carbon from the pyrolysis reaction may deposit on substrates within methane pyrolysis system 106.

In some examples, system 100 may be configured to divert at least a portion of generated carbon dioxide from methane generation. For example, in the absence of methane pyrolysis on a vehicle, there may not be enough hydrogen gas available to reduce all the carbon dioxide collected to methane. As a result, some carbon dioxide may be collected or vented to space, with the Sabatier reaction limited by hydrogen availability.

Oxygen generation system 108 is configured to generate oxygen and hydrogen from water, such as through electrolysis. In some examples, such as illustrated in FIG. 1A, oxygen generation system 108 may be configured to generate oxygen and hydrogen from water generated from the Sabatier reaction. As described above, the Sabatier reaction produces water, and at least a portion of this water may be used for oxygen and hydrogen generation. In other examples, oxygen generation system 108 may be configured to generate oxygen and hydrogen from water in the liquid sorbent. For example, as described in U.S. Pat. No. 11,466,372 entitled “ELECTROCHEMICAL CARBON DIOXIDE CONVERTER AND LIQUID REGENERATOR,” incorporated by reference in its entirety, electrolysis in a liquid sorbent, such as an ionic liquid sorbent, may involve electrochemically converting water in the liquid sorbent directly to oxygen and hydrogen. While the example of FIG. 1A describes generated methane and water as being used to generate hydrogen, in other examples, the generated methane and/or water may be used for other purposes, such as combustion or life support, or may be discharged to an atmosphere.

While system 100 is described with respect to methane generation system 104, system 100 may include other contaminant reduction systems. Such other contaminant reduction systems may utilize a removal stage that includes a liquid sorbent to absorb and desorb a contaminant and a subsequent reaction stage that includes a reactor to directly receive and reduce the contaminant. As a result, a contaminant may be removed from an atmosphere and reduced to at least partially recover a usable element or compound of the contaminant.

FIG. 1B is a block diagram illustrating an example methane generation system 104 for generating methane from carbon dioxide removed from an air stream. Methane generation system 104 is configured to remove at least a portion of the contaminants in the air stream using one or more liquid sorbents. A liquid sorbent may include any liquid configured to absorb and desorb a gaseous species. Liquid sorbents may be used with membrane contactors that contact an air stream with or draw an air stream from the liquid sorbent across one or more hydrophobic porous membranes.

Methane generation system 104 includes one or more scrubbers 110. Scrubber 110 is configured to absorb one or more contaminants from an air stream (“CABIN AIR”) into a liquid sorbent. The one or more contaminants include carbon dioxide (CO₂) and other contaminants, such as water vapor (H₂O). Methane generation system 104 includes one or more strippers downstream of scrubber 110. Stripper 112 is configured to desorb the one or more contaminants from the liquid sorbent and discharge an air stream (“CLEAN AIR”) having a lower concentration of contaminants than the received air stream. Loaded liquid sorbent (LS_{L}) that includes a relatively high concentration of contaminants is transferred from scrubber 110 to stripper 112, and unloaded liquid sorbent (LS_{U}) that includes a relatively low concentration of contaminants is transferred from stripper 112 to scrubber 110.

A contaminant stream desorbed by and discharged from stripper 112 may be at a relatively low pressure, and may include contaminants, such as water vapor, which may inhibit a Sabatier reaction. Methane generation system 104 includes a conditioning assembly 114 configured to condition the contaminant stream desorbed from stripper 112. Conditioning includes separating various contaminants other than carbon dioxide, such as water, such that the contaminant stream includes substantially only carbon dioxide, and bringing the carbon dioxide up to conditions for a Sabatier reaction. For example, the conditions of the Sabatier reaction, in combination with flow conditions and a composition of a catalyst, may be selected for a relatively high carbon dioxide conversion and methane selectivity. Such conditions may include a temperature between about 250 degrees Celsius (° C.) and about 450° C., and a pressure between about 55 kilopascals (kPa) and about 100 kPa. Conditioning assembly 114 includes a vacuum pump 118 configured to pressurize the carbon dioxide to a reaction pressure of the Sabatier reaction. Vacuum pump 118 may be configured to draw a vacuum on stripper 112 to lower a partial pressure of contaminants on a vapor side of a membrane of stripper 112 and increase a partial pressure gradient of the contaminants across the membrane.

Methane generation system 104 includes a Sabatier reactor 116 downstream of conditioning assembly 114, including vacuum pump 118. Sabatier reactor 116 is configured to directly (e.g., without intermediate long-term storage) receive carbon dioxide from conditioning assembly 114 and hydrogen from a hydrogen source, such as methane pyrolysis system 106 or oxygen generation system 108 of FIG. 1A. For example, Sabatier reactor 116 may directly receive the contaminant stream from conditioning assembly 114, such that Sabatier reactor 116 may continuously receive carbon dioxide from stripper 112 via conditioning assembly 114 while contaminants are desorbed by stripper 112. As a result, a flow rate of carbon dioxide from stripper 112 may be substantially equal to (e.g., within about 5% mass flow) a flow rate of carbon dioxide going into Sabatier reactor 116 Sabatier reactor 116 is configured to generate methane from carbon dioxide and hydrogen, such as by maintaining a temperature and pressure of the Sabatier reaction.

By incorporating the removal, conditioning, and processing of carbon dioxide into methane in a single system, methane generation system 104 may use less power than systems that do not remove carbon dioxide using a liquid sorbent or that store carbon dioxide prior to generating methane. As one example, vacuum pump 118 may be configured to pressurize carbon dioxide to a reaction pressure that is lower than a storage pressure, such as a reaction pressure of less than about 100 kilopascals (kPa). Removal, conditioning, and reduction of carbon dioxide may be controlled as a single continuous process that may not rely on batch or discontinuous processes, such as carbon dioxide storage, thereby simplifying control of the supply of carbon dioxide and reducing overall complexity of the system. Heat generated from the Sabatier reaction in Sabatier reactor 116 may be at least partially recovered by the liquid sorbent to assist in desorbing contaminants from the liquid sorbent at stripper 112. Methane generation system may have lower mass and volume compared to a system having the separate, unintegrated contaminant removal and reaction systems. Methane generation system 104 may have a greater tolerance to humidity, and corresponding reduced water separation requirements, compared to systems that include pressurizing the contaminants to a higher pressure for storage. Such greater tolerance for humidity may further reduce cooling power and reduce size and weight for water separation.

FIG. 1C is a schematic diagram illustrating an example contaminant processing system that includes methane generation system 104 for generating methane from carbon dioxide. System 104 includes a cabin air circuit (not labeled) configured to circulate cabin air between cabin 102 and scrubber 110 via a membrane dehumidifier 126. In the example of FIG. 1C, a cabin air stream 120 includes a filter 122 configured to remove particulates from cabin air stream 120 prior to entry into scrubber 110 and a blower 124 configured to draw cabin air into scrubber 110, while clean air stream 132 includes a filter 134 configured to remove any leaked liquid sorbent and/or further filter clean air from clean air stream 132 prior to entry into cabin 102.

In the example of FIG. 1C, system 104 includes membrane dehumidifier 126 to capture humidity from cabin air stream 120 to recover humidity into clean air stream 132 that may otherwise be absorbed by the liquid sorbent at scrubber 110. For example, membrane dehumidifier 126 may be positioned between cabin 102 and scrubber 110, such that cabin air received by scrubber 110 may include a lower humidity than cabin air received by system 104 from cabin 102. On one side, membrane dehumidifier 126 may be configured to receive cabin air stream 120 as a feed gas stream and discharge cabin air in a dehumidified air stream 128 to scrubber 110 having a lower humidity. On an opposite side, membrane dehumidifier 126 may be configured to receive a dehumidified clean air stream 130 from scrubber 110 and discharge clean air to clean air stream 132 having a higher humidity. By capturing humidity from cabin air prior to entry of the cabin air from cabin air stream 120 into scrubber 110, a greater amount of humidity may be preserved. Removing water prior to going through scrubber 110 may also reduce an amount of water that is absorbed into the liquid sorbent and, correspondingly, reduce an amount of water that may be removed by stripper 112 through evaporative cooling. This water removal by membrane dehumidifier 126 may permit smaller sizing of scrubber 110 and/or stripper 112, and/or a smaller load on pump 142 due to reduced volume, and/or may reduce an amount of power for heater 140 due to reduced evaporative cooling.

Additionally or alternatively, this water removal by membrane dehumidifier 126 may add tolerance to higher humidity in the cabin air and permit lower operating temperatures of scrubber 110 and, correspondingly, increasing carbon dioxide removal rates. For example, some cabins may have high humidity all or most of the time, or may have periods of high humidity. If the dew point of the air entering scrubber 110 is higher than a temperature of scrubber 110, humidity from the air may condense on a membrane of scrubber 110 on the gas side, which inhibits carbon dioxide transfer through the pores.

Methane generation system 104 is configured to remove at least a portion of the contaminants in the air stream using one or more liquid sorbents. A liquid sorbent may include any liquid configured to absorb and desorb a gaseous species. Liquid sorbents may be water soluble, hygroscopic (i.e., capable of absorbing moisture from the air), capable of absorbing or desorbing contaminants in response to a change in solubility driven by a change in temperature, and/or capable of releasing water by evaporation, such as by elevating the temperature or reducing the water partial pressure. In some examples, the liquid sorbent may be an ionic liquid sorbent. Ionic liquid sorbents may be salts that are generally comprised of an anion and an organic cation. These salts may be liquid at their temperature of use, have effectively zero vapor pressure, be generally nontoxic, and/or have sufficient stability to resist deterioration. In some examples, ionic liquid sorbents may contain relatively large organic cations and any of a variety of anions, which may be tailored to obtain desired characteristics, such as characteristics that improve absorption of the particular contaminant under operating conditions of methane generation system 104. A variety of ionic liquid sorbents may be used including, but not limited to, imidazolium salts, such as 1-ethyl-3-methylimidazolium (EMIM) acetate (Ac).

In system 104, the liquid sorbent is dissolved in water to form a liquid sorbent mixture. A concentration of liquid sorbent in the liquid sorbent mixture may be sufficiently high to remove a particular or set of contaminants and sufficiently low that the liquid sorbent remains in solution through operating ranges (e.g., temperature range, pH range) and/or maintains a low viscosity for maintaining high mass transfer. In some examples, the liquid sorbent mixture may further include a dissolved promoter. The promoter may be configured to increase a rate of removal of a contaminant, such as water or carbon dioxide, from an air stream. For example, the promoter may be configured to reduce a viscosity of the liquid sorbent, change a pH of the liquid sorbent, increase a thermal stability of the liquid sorbent, increase a capacity of the liquid sorbent for the contaminant, or increase an absorption rate of the contaminant into the liquid sorbent. Absorption of the contaminants by the liquid sorbent may be determined by a concentration of the contaminants in the corresponding air stream. Liquid sorbents may be used with membrane contactors that contact an air stream with or draw an air stream from the liquid sorbent across one or more hydrophobic porous membranes.

Scrubbers 110 and/or strippers 112 described herein may include one or more membrane separators configured to flow air on a first side and flow liquid sorbent on a second, opposite side. For example, a membrane separator may include a plurality of parallel membrane contactors. A membrane contactor may include a cylindrical module filled with parallel or woven hollow porous fibers forming a hydrophobic porous membrane. For example, dimensions of these hollow fibers could be less than about 3 mm, and the pore dimension could be less than about 2 microns. The high surface area of the hollow fiber membrane contactors enables a high mass transfer of contaminant gases, such as carbon dioxide and water, into the respective liquid sorbent using a relatively small system volume and weight. The material of the hollow fibers can be selected such that the liquid sorbent does not wet the pores, and the trans-membrane pressure is kept sufficiently low to prevent pore penetration. As a result, the membrane contactor may ensure that the liquid sorbent and gas stream do not need further separation, such that methane generation system 104 may act in a gravity-independent way without the use of moving parts. Fiber materials may include, but are not limited to, hydrophobic materials such as polypropylene, polyvinylidene fluoride, polysulfone, polyimide, polytetrafluoroethylene (PTFE), and the like. In some examples, a coating may be applied to reduce liquid flow through the pores. Coatings that may be used include, but are not limited to, PTFE, a crosslinked siloxane, perfluorinated polymers, functionalized nanoparticles, and the like to prevent liquid flow through the pores. While described in FIG. 1C as flowing through a “tube” side, liquid sorbent flow can be either on the “tube” side or the “shell” side, while gas is flowed on the opposite side.

Scrubber 110 is configured to absorb one or more contaminants from cabin air stream 120 into the liquid sorbent and discharge a clean air stream 132 to cabin 102. Clean air stream 132 has a lower concentration of contaminants than cabin air stream 120. For example, clean air stream 132 may have a concentration of carbon dioxide that is about 25% to about 99% less than a concentration of carbon dioxide in cabin air stream 120. Scrubber 110 includes one or more separation membranes, each configured to flow (e.g., provide or direct flow of) cabin air from cabin air stream 120 on a gas phase side (e.g., a tube side) of the respective membrane and flow the liquid sorbent on a liquid phase side (e.g., a shell side) of the membrane.

On a gas phase side, scrubber 110 is configured to receive cabin air from cabin air stream 120 that includes contaminants from cabin 102. Contaminants may pass through the membrane due to a concentration gradient between the cabin air and the liquid sorbent and become absorbed by the liquid sorbent, while the liquid sorbent may not substantially flow through the membrane. As a result, clean air from clean air stream 132 discharged from scrubber 110 may have a lower concentration of contaminants than cabin air from cabin air stream 120 received by scrubber 110. Scrubber 110 is configured to discharge clean air stream 132 to cabin 102 via membrane dehumidifier 126. On a liquid phase side, scrubber 110 is configured to receive unloaded liquid sorbent, such as from a liquid sorbent storage 146. The unloaded liquid sorbent may flow through scrubber 110 and absorb carbon dioxide and other gaseous contaminants from cabin air through the membrane(s) of scrubber 110. As a result, the loaded liquid sorbent discharged from scrubber 110 may have a higher concentration of contaminants than the unloaded liquid sorbent received by scrubber 110. Scrubber 110 may discharge the loaded liquid sorbent containing the contaminants to stripper 112.

Stripper 112 is configured to desorb the contaminants, including carbon dioxide, from the liquid sorbent into contaminant stream 148. Stripper 112 includes one or more separation membranes, each configured to collect (e.g., receive through the membrane) contaminants on a gas phase side (e.g., a tube side) of the respective membrane and flow the loaded liquid sorbent on a liquid phase side (e.g., a shell side) of the membrane. On a liquid phase side, stripper 112 is configured to receive loaded liquid sorbent from scrubber 110 and desorb contaminants from the loaded liquid sorbent. Contaminants may flow across fibers of the membrane due to a concentration gradient, while the liquid sorbent may not substantially flow across the fibers of the membrane. As a result, unloaded liquid sorbent discharged from stripper 112 may have a lower concentration of contaminants than the loaded liquid sorbent received by stripper 112. On a gas phase side, stripper 112 is configured to discharge the contaminants in contaminant stream 148. Contaminant stream 148 may be continuously removed from stripper 112 to assist migration of the contaminants from the loaded liquid sorbent into contaminant stream 148.

System 104 includes liquid sorbent circuit 136 configured to circulate liquid sorbent between scrubber 110 and stripper 112. For example, a pump 142 may pump unloaded liquid sorbent from stripper 112 into scrubber 110. Unloaded liquid sorbent may include unused liquid sorbent free of contaminants or regenerated liquid sorbent having a lower concentration of contaminants than the loaded liquid sorbent. Liquid sorbent storage 146 may store liquid sorbent, such as in a relatively cool state.

Liquid sorbent circuit 136 may include one or more components for controlling a temperature of the liquid sorbent. In some examples, the unloaded liquid sorbent may be cooled by a regenerative heat exchanger 138 and/or a heat exchanger 144 prior to entry into scrubber 110. Heat exchanger 138 is configured to exchange heat between a relatively hot unloaded liquid sorbent from stripper 112 and a relatively cool loaded liquid sorbent from scrubber 110. Heat exchanger 144 configured to receive the unloaded liquid sorbent from stripper 112, cool the unloaded liquid sorbent, and discharge the cooled, unloaded liquid sorbent to scrubber 110. Liquid sorbent circuit 136 may include one or more heaters 140 upstream of stripper 112. Heaters 140 may be configured to heat the liquid sorbent prior to entry into stripper 112 to increase desorption of contaminants from the liquid sorbent.

In the example of FIG. 1C, system 104 may include one or more systems or components configured to further process contaminant stream 148, such that carbon dioxide may be isolated and pressurized for reaction in Sabatier reactor 116. System 104 includes Sabatier reactor 116 configured to generate hydrocarbons using carbon dioxide removed by scrubber 110. Sabatier reactor 116 may require a water concentration of less than 4% to react hydrogen with carbon dioxide. However, in a life support application, a large amount of water may be present in cabin air stream 120.

System 104 includes vacuum pump 118, condenser 154, and water separator 156 configured to pressurize contaminant stream 148 and remove water from the compressed contaminant stream. For example, for carbon dioxide removed from stripper 112 to be reacted efficiently by Sabatier reactor 116, vacuum pump 118, condenser 154, and water separator 156 may pressurize contaminant stream 148 to a moderate pressure and remove nearly all water from contaminant stream 148. However, this pressure may be substantially lower than a pressure at which carbon dioxide would otherwise be stored at. In some examples, system 104 includes a filter 150 configured to remove entrained liquid sorbent from contaminant stream 148.

Vacuum pump 118 is configured to pressurize contaminant stream 148 and draw a vacuum on stripper 112. Vacuum pump 118 is configured to pressurize the carbon dioxide to a reaction pressure. In some examples, the reaction pressure is less than about 100 kilopascals (kPa). For example, vacuum pump 118 may be configured to operate at a pressure between about 50 kPa and about 100 kPa. A variety of vacuum pumps may be used for vacuum pump 118 including, but not limited to, centrifugal compressors, positive displacement compressors, and the like.

System 104 includes a condenser 154 configured to condense desorbed water from the carbon dioxide. Condenser 154 may be configured to cool contaminant stream 148 and condense water from contaminant stream 148. For example, condenser 154 may be coupled to a cooling medium system or other cooling system that circulates a cooling medium to cool contaminant stream 148. A variety of condensers may be used for condenser 154 including, but not limited to, shell and tube heat exchangers, plate-fin, surface coolers, heat pipes, thermoelectric devices, cooling jackets, and the like.

Water separator 156 is positioned upstream of Sabatier reactor 116 and is configured to separate desorbed water from the carbon dioxide. Water separator 156 may be configured to remove water from contaminant stream 148, discharge a carbon dioxide stream 158 to Sabatier reactor 116, and discharge water condensate stream 160 to water storage 162. A variety of water separators may be used for water separator 156 including, but not limited to, static phase separators, capillary phase separator, membrane phase separators, centrifugal/rotary separators, and the like.

Sabatier reactor 116 is configured to generate methane from the carbon dioxide and, to a lesser degree, carbon monoxide. Sabatier reactor 116 includes a catalyst configured to increase a reaction rate of the Sabatier reaction. In some examples, a structure of the catalyst may be configured to further increase thermal and/or mass transfer of reactants and products. For example, Sabatier reactor 116 may include a catalyst in the form of any of a mesh, a packed bed, a microchannel grid, or other structure or combination of structures having a high surface area, high thermal conductivity, and/or high reactant throughput. Catalysts that may be used include, but are not limited to, nickel, ruthenium, rhodium, and the like, alone or on a support, such as aluminum oxide.

In some examples, Sabatier reactor 116 is configured to operate at a relatively high carbon dioxide conversion (e.g., >70%) and methane selectivity (e.g., >80%) at relatively low temperatures and pressures. For example, the catalyst may be configured to achieve such high carbon dioxide conversion and methane selectivity at temperatures less than about 450° C. and/or pressures less than about 100 kPa. A pressure within Sabatier reactor 116 may be sufficient to condense water so that water is not lost downstream, such as a pressure greater than about 50 kPa. On the other hand, maintaining a pressure at less than 100 kPa may create a vacuum in Sabatier reactor 116, such that methane or other combustibles may not leak out of Sabatier reactor 116 into a cabin space. Further, maintaining a low pressure may reduce an amount of power used by vacuum pump 118.

System 104 may further include post-processing components configured to further separate products from Sabatier reactor 116. System 104 includes a condenser 164 configured to receive the products in a product stream 174, including methane and water vapor, from Sabatier reactor 116 and cool the products to condense water and purify the methane. System 104 includes a water separator 166 downstream of Sabatier reactor 116 and configured to separate the generated water from the methane. The separated water may be discharged in a post-reaction water stream 170 to water storage 162. The resulting methane stream 168 may have a high concentration of methane (e.g., >95%), and may be vented or discharged to another system. For example, the methane may be vented to atmosphere, discharged to a storage system, or used in another process, such as methane pyrolysis.

Sabatier reactor 116 may generate heat during the Sabatier reaction, such that products of Sabatier reactor 116, such as methane and water vapor, may be at relatively high temperatures. System 104 may be configured to recycle at least a portion of the heat generated from Sabatier reactor 116 through a thermal link circuit 172. Thermal link circuit 172 is configured to transfer at least a portion of heat from either Sabatier reactor 116 or product stream 174 to liquid sorbent circuit 136 upstream of stripper 112. Thermal link circuit 172 may include a blower 186 or other pressure source (e.g., a pump) configured to circulate a heat transfer medium between Sabatier reactor 116, one or more heat exchangers 182 in liquid sorbent circuit 136, and/or one or more heat exchangers 184 downstream of Sabatier reactor 116. The heat transfer medium may remove heat from Sabatier reactor 116 and/or product stream 174 generated during the Sabatier reaction. Heat exchanger 182 may be configured to receive the relatively hot heat transfer medium and heat the liquid sorbent. As a result, the liquid sorbent may be heated prior to being received by stripper 112, thereby reducing or eliminating a heat load on heater 140. Heat exchanger 184 may be configured to receive the relatively cool heat transfer medium from heat exchanger 182 and cool the product stream from Sabatier reactor 116. As a result, a product stream from Sabatier reactor 116 may be cooled prior to being processed and/or stored, thereby reducing or eliminating a cooling load on condenser 154.

In some examples, thermal link circuit 172 may be a closed loop separate from product stream 174. For example, the liquid sorbent in liquid sorbent circuit 136 may be sensitive to high localized temperatures, such that a temperature of product stream 174 exiting Sabatier reactor 116 may result in degradation of the liquid sorbent. Rather than directly interface product stream 174 with the liquid sorbent at heat exchanger 182, thermal link circuit 172 may be configured to circulate a separate heat transfer medium between heat exchanger 182 and Sabatier reactor 116 and/or heat exchanger 184. The heat transfer medium may include any fluid configured to absorb heat from Sabatier reactor 116 and/or product stream 174 and transfer heat to the ionic liquid in liquid sorbent circuit 136.

While illustrated in FIG. 1C as circulating a separate heat transfer medium through thermal link circuit 174, in some examples, thermal link circuit 172 may be replaced with a regenerative heat exchanger for heat exchanger 182, and the heat transfer medium is a portion of product stream 174. For example, a contaminant reduction system that either uses a different liquid sorbent that can maintain thermal stability at the temperature of product stream 174, or involves a different reaction that results in a lower temperature of product stream 174, may be configured to directly interface product stream 174 with liquid sorbent circuit 136 through heat exchanger 182. In such instances, product stream 174 may directly flow to heat exchanger 182, and subsequently flow from heat exchanger 182 to condenser 164, to cool product stream 174 and heat liquid sorbent in liquid sorbent circuit 136.

Various thermal components in system 104 may be cooled using an external cooling source, such as cooling medium system 178. System 104 includes a coolant loop 176 configured to remove heat from heat exchanger 144, condenser 154, and condenser 164. In the example of FIG. 1C, condensers 164 and 154 and heat exchanger 144 are arranged in series according to a desired temperature of a cooling medium encountering each fluid stream to achieve the desired degree of cooling. For example, it is important to condense out as much water as possible in product stream 174 (at condenser 164) and contaminant stream 148 (at condenser 154), as any water remaining may be a problem for downstream processes/storage or may be lost to space vacuum. The colder the cooling fluid, and correspondingly the higher the temperature difference between the cooling fluid and the cooled stream in the corresponding condenser, the more water can be condensed. Cooling the ionic liquid may be important for the efficiency of contaminant removal, and can still be achieved after some heating of the cooling medium from other processes, and may not impact product purity to downstream processes/material loss to space. Coolant loop 176 is configured to circulate the cooling medium through various heat exchangers, such as heat exchanger 144, condenser 154, and condenser 164, to cool liquid sorbent (as in heat exchanger 144), contaminant stream 148 (as in condenser 154), and/or product stream 174 (as in condenser 164).

System 104 may include a control system (not labeled) communicatively coupled to and configured to receive measurement signals from one or more sensor sets, and other process control components (not shown) of system 104, such as: control valves for cabin air stream 120, clean air stream 132, contaminant stream 148, product stream 174, and inlets/outlets to heat exchangers 138 and 144, heater 140, liquid sorbent storage 146; pump 142; blower 124, vacuum pump 118; pumps of cooling medium system 178; and the like. The control system is configured to control a concentration of one or more contaminants within the environment of cabin 102. For example, the control system may be configured to receive a concentration measurement for a contaminant, such as carbon dioxide, such as from a cabin air sensor set or a carbon dioxide concentration sensor in cabin 102. The control system may be configured to determine whether the concentration measurement of the contaminant exceeds a concentration setpoint. For example, the concentration setpoint may be a target concentration of the contaminant for maintaining cabin 102 below a threshold contaminant concentration. The control system may be configured to send, in response to the concentration measurement of the contaminant exceeding the concentration setpoint, a control signal to decrease a concentration of the contaminant in an air stream returned to cabin 102. For example, the control system may send a control signal to control a flow rate of the liquid sorbent mixture; a temperature of the liquid sorbent mixture at scrubber 110 or stripper 112; a flow rate of the cabin air stream from cabin 102; or any other variable that may control a rate of removal of the contaminant from the cabin air stream from cabin 102.

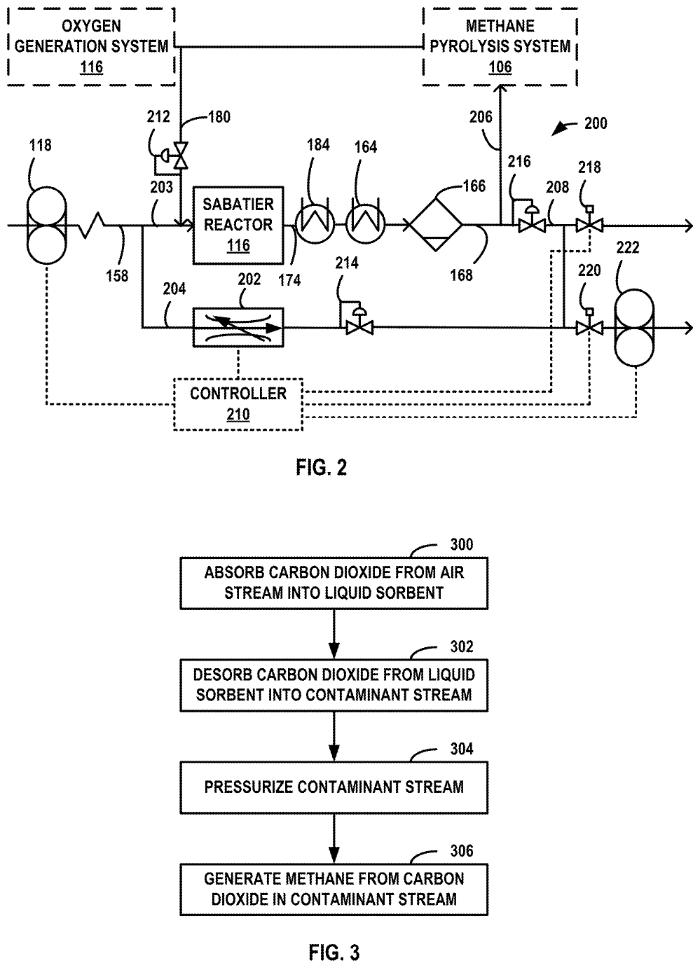

FIG. 2 is a schematic diagram illustrating an example Sabatier assembly 200 of a methane generation system, such as methane generation system 104 of FIGS. 1A-1C. Assembly 200 includes a variety of components configured to control a Sabatier reaction using carbon dioxide desorbed from a liquid sorbent. As such, assembly 200 may include any components that may be configured to measure conditions or control parameters related to a composition or flow of a contaminant stream, a composition or flow of a reactant stream, or a composition or flow of a product stream related to the Sabatier reaction. While FIG. 2 will be described with respect to a Sabatier reaction, assembly 200 may be used to control other reduction reactions, such as a Bosch reaction, that received contaminants desorbed from a liquid sorbent.

Assembly 200 includes various components for controlling flow of reactants into and around, and products from, Sabatier reactor 116. For example, system 104 of FIGS. 1A-1C may be configured to remove carbon dioxide from a cabin air stream to maintain the carbon dioxide within a desired threshold. However, a flow rate of various other reactants used in the Sabatier reaction, such as hydrogen gas, may not be sufficient or desired to react with all the carbon dioxide. To handle a continuous, and possibly fluctuating, stream of contaminants, assembly 200 includes a Sabatier branch for discharging carbon dioxide to Sabatier reactor 116 and a bypass branch for diverting carbon dioxide around Sabatier reactor 116. Assembly 200 includes a carbon dioxide flow controller 202 downstream of stripper 112 in the bypass branch. Carbon dioxide flow controller 202 is configured to control a flow rate of a carbon dioxide bypass stream 204 around Sabatier reactor 116, with a remainder of carbon dioxide stream 158 flowing into Sabatier reactor 116. Placement of flow controller 202 in the bypass branch may assist in maintaining a low pressure drop in the Sabatier branch.

Carbon dioxide bypass stream 204 may be either vented or stored. For example, assembly 200 may include a storage valve 220 configured to control a flow rate of carbon dioxide to storage and a compressor 222 configured to pressurize the carbon dioxide to a higher pressure for storage. A portion of carbon dioxide may be vented.

Product stream 168 may be split between a methane feed stream 206 flowing to a methane storage or processing system, such as methane pyrolysis system 106, and a methane vent stream 208. Assembly 200 may include a vent valve 218 configured to control venting of carbon dioxide bypass stream 204 and methane vent stream 208. Assembly 200 includes other components, such as vacuum pump 118, configured to assist in maintaining reaction conditions within Sabatier reactor 116.

Assembly 200 includes a process control system that includes a controller 210 and one or more sensor sets (not labeled). Controller 210 is configured to control reactant streams into and conditions within Sabatier reactor 116 to maintain a Sabatier reaction. Controller 210 may include any of a wide range of devices, including control circuitry, processors (e.g., one or more microprocessors, one or more application specific integrated circuits (ASICs), one or more field programmable gate arrays (FPGAs), or the like), processing circuitry, one or more servers, one or more desktop computers, one or more notebook (i.e., laptop) computers, one or more cloud computing clusters, or the like.

Controller 210 may be configured to receive measurements from the one or more sensor sets and/or components of assembly 200 and/or send control signals to components of assembly 200. Controller 210 may be communicatively coupled to and configured to receive measurement signals from the one or more sensor sets, and other process control components (not shown) of methane generation system 104, such as: control valves for various streams; pumps; heaters; heat exchangers; compressors; and the like. The sensor sets may include instrumentation configured to detect any of a pressure, temperature, flow rate, and/or contaminant concentration (e.g., carbon dioxide concentration or water concentration) of a liquid or gas stream of methane generation system 104. Controller 210 may be configured to use the detected conditions to control operation of assembly 200, and in some instances, other components of methane generation system 104, to function as described in the application.

Without being limited to any particular theory, a controller 210 may be configured to operate Sabatier reactor 116 by reacting carbon dioxide, and optionally carbon monoxide, with hydrogen to produce methane and water, as shown in Equations 1 and 2 below:

$${CO}_{2} + 4 {H}_{2} \to {CH}_{4} + 2 {H}_{2} O \qquad [ Equation 1 ]$$

$$CO + 3 {H}_{2} \to {CH}_{4} + {H}_{2} O \qquad [ Equation 2 ]$$

Ideally, controller 210 may operate Sabatier reactor 116 in a manner that encourages carbon dioxide and carbon monoxide methanation through a Sabatier reaction, rather than reduction through a reverse water gas shift (RWGS) or Bosch reaction. To achieve a high carbon dioxide conversion and methane selectivity, controller 210 may operate Sabatier reactor 116 at relatively low temperatures, such as less than about 450° C., and a relatively high ratio (e.g., greater than stoichiometric) of hydrogen to carbon dioxide.

In some examples, controller 210 is configured to maintain reaction conditions within Sabatier reactor 116 based on a flow rate of carbon dioxide. For example, a control system of system 104 may operate various components of liquid sorbent circuit 136 to remove carbon dioxide at a particular rate that maintains a concentration of contaminants in cabin 102 above, below, or within a range of one or more thresholds. In some instances, controller 210 is configured to remove carbon dioxide at a particular rate that corresponds to cabin needs, such as to maintain cabin 102 below a particular cabin concentration threshold. As a result, carbon dioxide may be desorbed from stripper 112 at a particular flow rate that may not correspond to flow rates of other reactants, such as hydrogen gas, in stoichiometric amounts. Carbon dioxide that is removed in excess of carbon dioxide required for methane generation may be stored, such as through storage valve 220 and compressor 222, or vented to atmosphere, such as through vent valve 218.

Controller 210 may be configured to control a reaction pressure and temperature of Sabatier reactor 116, among other parameters, such that Sabatier reactor 116 produces a product stream of methane and water. Controller 210 may receive measurement signals from a flow meter that indicates a flow rate of carbon dioxide from stripper 112. Based on the flow rate of carbon dioxide, controller 210 may send control signals to back pressure regulators 212, 214, and 216 to control a back pressure of hydrogen gas stream 180, carbon dioxide bypass stream 214, and methane stream 168, respectively. The product stream may have a concentration of carbon dioxide below a particular threshold associated with a target carbon dioxide conversion and/or a concentration of methane above a particular threshold associated with a target methane selectivity. For example, the threshold associated with a target carbon dioxide conversion may be greater than about 70% and/or the threshold associated with a target methane selectivity may be greater than about 90%.

In some examples, controller 210 is configured to maintain a reactant stream to and/or a product stream from Sabatier reactor 116 at relatively low concentrations of water vapor. For example, controller 210 may receive measurement signals indicating a concentration of water vapor in contaminant stream 148 and/or product stream 174, and send control signals to water separators 156 and/or 166 and cooling medium system 178 to control remove water vapor and maintain a target concentration of water vapor below a threshold in carbon dioxide stream 158 and/or methane stream 168. For example, a concentration of water in each of carbon dioxide stream 158 and/or methane stream 168 may be maintained less than about 10 vol. %, such as less than or equal to about 5 vol. % for carbon dioxide stream 158 and less than or equal to about 5 vol. % for methane stream 168.

FIG. 3 is an example flowchart of a method for generating methane from carbon dioxide. FIG. 3 will be described with respect to FIGS. 1B, 1C, and 2; however, other systems may be used to perform the method of FIG. 3. The method of FIG. 3 includes absorbing one or more contaminants from a cabin air stream into a liquid sorbent (300). For example, scrubber 110 of methane generation system 104 may be maintained at particular conditions, such as temperature and flow rate of liquid sorbent, to facilitate transfer of the one or more contaminants into the liquid sorbent, such as by controlling pump 142 to circulate the liquid sorbent between scrubber 110 and stripper 112, controlling heat exchanger 144 to cool the liquid sorbent prior to entry into scrubber 110, and/or controlling heat exchanger 138 to recover a portion of heat from liquid sorbent discharge from stripper 112. These particular conditions may be based on a particular measured or anticipated composition of cabin air stream 120.

The method of FIG. 3 includes desorbing the one or more contaminants from the liquid sorbent (302). For example, stripper 112 may be maintained at particular conditions, such as temperature and flow rate of liquid sorbent and vacuum from vacuum pump 118, to facilitate transfer of the one or more contaminants from the liquid sorbent, such as by controlling vacuum pump 118 to maintain a vacuum at stripper 112 or controlling heater 140 to heat the liquid sorbent prior to entry into stripper 112. These particular conditions may be based on a particular measured or anticipated composition of cabin air stream 120.

The method of FIG. 3 includes pressurizing the one or more contaminants in the contaminant stream (304). For example, vacuum pump 118 may be operated at particular conditions, such as pumping speed, to maintain a vacuum on stripper 112 and maintain a reaction pressure at Sabatier reactor 116, in combination with back pressure regulators 212, 214, 216. For example, vacuum pump 118 may be operated to pressurize contaminant stream 148 to a pressure from about 55 kPa to about 100 kPa.

The method of FIG. 3 includes generating methane from carbon dioxide (306). For example, carbon dioxide flow controller 202 may be operated at particular flow rates to maintain a particular composition of reactants, such as carbon dioxide and hydrogen, and products, such as methane and water vapor, in product stream 168. For example, carbon dioxide flow controller 202 may be operated to produce a flow rate of carbon dioxide such that product stream 174 has a concentration of carbon dioxide and methane associated with a carbon dioxide conversion greater than about 70% and a methane selectivity greater than about 90%.

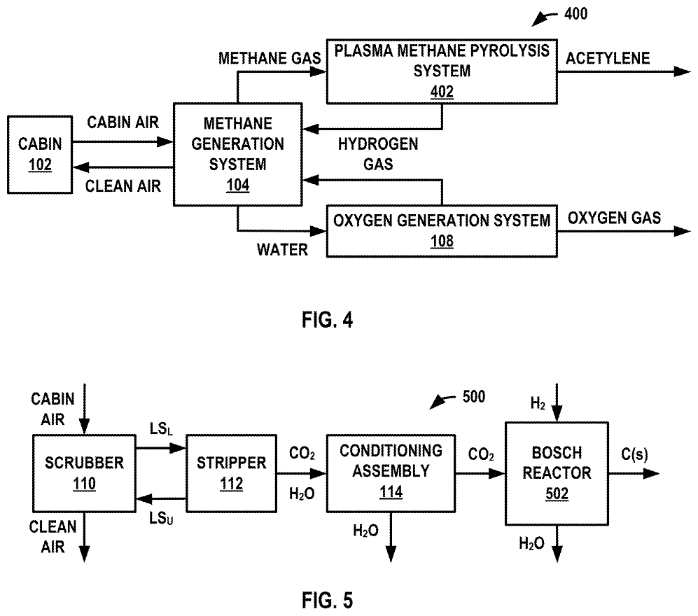

While FIG. 1A has been described with respect to a methane pyrolysis system, contaminant reduction systems described herein may be used in combination with any post-processing system that may further process a reduced contaminant into a useful form or to receive elements or compounds. FIG. 4 is a block diagram illustrating an example contaminant processing system 400 for producing oxygen and hydrocarbons from carbon dioxide. In the example of FIG. 4, system 400 includes a plasma processing system 402 for generating acetylene (and trace water, carbon monoxide, ethylene, ethane, and carbon) and hydrogen gas from methane gas. Plasma pyrolysis system 402 may be configured to receive methane gas from methane generation system 104 and generate hydrogen gas for further methane and water generation at methane generation system 104, as in Equation 3 below:

$$2 C {H}_{4} \to 3 {H}_{2} + {C}_{2} {H}_{2} \qquad [ Equation 3 ]$$

Plasma pyrolysis may involve electrical energy and be capable of starting up and shutting down relatively quickly. Further, compared to a Bosch reactor, a plasma methane pyrolysis reactor may be less susceptible to fouling and catalyst deactivation by carbon deposits, necessitating frequent catalyst regeneration or replacement of the catalyst, and less susceptible to plugging by deposition of carbon within fixed beds, with resulting increase of reactor efficiency due to less channeling of flow, lower pressure drops, and fewer limitations on mass transfer.

Contaminant reduction systems described herein may be used with any reaction for which a contaminant removed using a liquid sorbent may be reduced to a more useful form. In some examples, rather than reduce carbon dioxide using a Sabatier reaction, carbon dioxide removed from a cabin air stream may be reduced using a Bosch reaction. FIG. 5 is a block diagram illustrating an example carbon generation system for generating carbon from carbon dioxide. In the example of FIG. 5, system 500 includes a Bosch reactor 502 for generating solid carbon from carbon dioxide. Bosch reactor 502 may be configured to receive carbon dioxide from conditioning assembly 144 and hydrogen gas, and generate water and solid carbon, as in Equations 4 (reverse water gas shift reaction), Equation 5 (carbon monoxide hydrogenation reaction), Equation 6 (Boudouard reaction), and Equation 7 (net Bosch reaction):

$${CO}_{2} + {H}_{2} \to {H}_{2} O + CO \qquad [ Equation 4 ]$$

$$CO + {H}_{2} \to {H}_{2} O + C ( s ) \qquad [ Equation 5 ]$$

$$2 CO \to {CO}_{2} + C ( s ) \qquad [ Equation 6 ]$$

$${CO}_{2} + 2 {H}_{2} \to 2 {H}_{2} O + C ( s ) \qquad [ Equation 7 ]$$

In contrast to the Sabatier reaction, which may involve intermediate methane generation and subsequent pyrolysis to produce solid carbon, Bosch reactor 502 may be configured to generate solid carbon, either directly or through a series of reactors.

While systems have been described with respect to contaminant reduction systems, systems may also be used with any reaction for which a contaminant removed using a liquid sorbent may be oxidized to a more useful form. In some examples, rather than reduce contaminants using a reduction reaction, contaminants removed from a cabin air stream may be oxidized using an oxidation reaction.

Example 1: A system for generating methane includes a scrubber configured to absorb one or more contaminants from an air stream into a liquid sorbent, wherein the one or more contaminants comprise carbon dioxide; a stripper configured to desorb the one or more contaminants from the liquid sorbent; a vacuum pump configured to pressurize the desorbed contaminants to a reaction pressure of the carbon dioxide; a water separator configured to remove water from the pressurized, desorbed contaminants; and a Sabatier reactor configured to generate the methane from the carbon dioxide of the pressurized, desorbed contaminants.

Example 2: The system of example 1, wherein the reaction pressure is less than 100 kilopascals (kPa).

Example 3: The system of any of examples 1 and 2, wherein the vacuum pump is configured to draw a vacuum on the stripper.

Example 4: The system of any of examples 1 through 3, further includes a heat exchanger configured to receive the liquid sorbent from the scrubber and discharge the liquid sorbent to the stripper; and a thermal link circuit fluidically coupled to the heat exchanger and the Sabatier reactor, wherein the thermal link circuit is configured to flow a heat transfer medium heat the liquid sorbent using heat generated by the Sabatier reactor.

Example 5: The system of example 4, wherein the heat exchanger is a first heat exchanger, wherein the system further comprises a second heat exchanger configured to receive the methane from the Sabatier reactor; and wherein the thermal link circuit is fluidically coupled to the second heat exchanger and configured to flow the heat transfer medium from the first heat exchanger to the second heat exchanger to cool the methane.

Example 6: The system of any of examples 1 through 5, further includes receive the liquid sorbent from the scrubber, heat the liquid sorbent using at least a portion of the methane from the Sabatier reactor; and discharge the liquid sorbent to the stripper.

Example 7: The system of any of examples 1 through 6, further includes a first condenser configured to receive the methane from the Sabatier reactor and condense generated water from the methane; a second condenser configured to receive the carbon dioxide from the stripper and condense desorbed water from the carbon dioxide; a heat exchanger configured to receive the liquid sorbent from the stripper and discharge the liquid sorbent to the scrubber; and a coolant loop configured to remove heat from the first condenser, the second condenser, and the heat exchanger in series.

Example 8: The system of any of examples 1 through 7, further comprising a methane pyrolysis reactor configured to generate hydrogen from pyrolysis of the methane.

Example 9: The system of any of examples 1 through 8, further comprising an oxygen generation assembly configured to generate oxygen from electrolysis of generated water.

Example 10: The system of any of examples 1 through 9, wherein the pressurized, desorbed contaminants are split between a contaminant feed stream to the Sabatier reactor and a bypass stream, and wherein the bypass stream comprises a carbon dioxide flow controller configured to control a flow rate of the contaminant feed stream into the Sabatier reactor by controlling flow rate of the bypass stream.

Example 11: A method for generating methane includes absorbing, by a scrubber, one or more contaminants from an air stream into a liquid sorbent, wherein the one or more contaminants comprise carbon dioxide; desorbing, by a stripper, the one or more contaminants from the liquid sorbent; pressurizing, by a vacuum pump, the desorbed contaminants to a reaction pressure of the carbon dioxide; removing, by a water separator, water from the pressurized, desorbed contaminants; and generating, by a Sabatier reactor, the methane from the carbon dioxide of the pressurized, desorbed contaminants.

Example 12: The method of example 11, wherein the reaction pressure is less than 100 kilopascals (kPa).

Example 13: The method of any of examples 11 and 12, wherein the vacuum pump is configured to draw a vacuum on the stripper.

Example 14: The method of any of examples 11 through 13, further comprising heating, by a thermal link circuit using a heat transfer medium, the liquid sorbent with heat generated by the Sabatier reactor.

Example 15: The method of any of examples 11 through 14, further comprising heating, by a heat exchanger using the methane from the Sabatier reactor, the liquid sorbent with heat generated by the Sabatier reactor.

Example 16: The method of any of examples 11 through 15, further includes receiving, by a first condenser, the methane from the Sabatier reactor; receiving, by a second condenser, the carbon dioxide from the stripper; receiving, by a second heat exchanger, the liquid sorbent from the stripper; and removing, by a coolant loop, heat from the first condenser, the second condenser, and the heat exchanger in series.

Example 17: The method of any of examples 11 through 16, further comprising generating, by a methane pyrolysis reactor, hydrogen from pyrolysis of the methane.

Example 18: The method of any of examples 11 through 17, further generating, by an oxygen generation assembly, oxygen from electrolysis of generated water.

Example 19: The method of any of examples 11 through 18, wherein generating the methane comprises controlling, by a control system, a rate of reaction of the Sabatier reactor based on at least one of a concentration of carbon dioxide in a contaminant stream discharged by the stripper or a flow rate of hydrogen entering the Sabatier reactor.

Example 20: The method of example 19, wherein the pressurized, desorbed contaminants are split between a contaminant feed stream to the Sabatier reactor and a bypass stream, and wherein controlling the rate of reaction of the Sabatier reactor comprises controlling a carbon dioxide flow controller to control a flow rate of the contaminant feed stream into the Sabatier reactor by controlling flow rate of the bypass stream.

Various examples have been described. These and other examples are within the scope of the following claims.

What is claimed is:

1. A system for generating methane, comprising:
a scrubber configured to absorb one or more contaminants from an air stream into a liquid sorbent, wherein the one or more contaminants comprise carbon dioxide;
a stripper configured to desorb the one or more contaminants from the liquid sorbent;
a vacuum pump configured to pressurize the desorbed contaminants to a reaction pressure of the carbon dioxide;
a water separator configured to remove water from the pressurized, desorbed contaminants; and
a Sabatier reactor configured to generate the methane from the carbon dioxide of the pressurized, desorbed contaminants.

2. The system of claim 1, wherein the reaction pressure is less than 100 kilopascals (kPa).

3. The system of claim 1, wherein the vacuum pump is configured to draw a vacuum on the stripper.

4. The system of claim 1, further comprising:
a heat exchanger configured to receive the liquid sorbent from the scrubber and discharge the liquid sorbent to the stripper; and
a thermal link circuit fluidically coupled to the heat exchanger and the Sabatier reactor, wherein the thermal link circuit is configured to flow a heat transfer medium heat the liquid sorbent using heat generated by the Sabatier reactor.

5. The system of claim 4,
wherein the heat exchanger is a first heat exchanger,
wherein the system further comprises a second heat exchanger configured to receive the methane from the Sabatier reactor; and
wherein the thermal link circuit is fluidically coupled to the second heat exchanger and configured to flow the heat transfer medium from the first heat exchanger to the second heat exchanger to cool the methane.

6. The system of claim 1, further comprising a heat exchanger configured to:
receive the liquid sorbent from the scrubber,
heat the liquid sorbent using at least a portion of the methane from the Sabatier reactor; and
discharge the liquid sorbent to the stripper.

7. The system of claim 1, further comprising:
a first condenser configured to receive the methane from the Sabatier reactor and condense generated water from the methane;
a second condenser configured to receive the carbon dioxide from the stripper and condense desorbed water from the carbon dioxide;
a heat exchanger configured to receive the liquid sorbent from the stripper and discharge the liquid sorbent to the scrubber; and
a coolant loop configured to remove heat from the first condenser, the second condenser, and the heat exchanger in series.

8. The system of claim 1, further comprising a methane pyrolysis reactor configured to generate hydrogen from pyrolysis of the methane.

9. The system of claim 1, further comprising an oxygen generation assembly configured to generate oxygen from electrolysis of generated water.

10. The system of claim 1,
wherein the pressurized, desorbed contaminants are split between a contaminant feed stream to the Sabatier reactor and a bypass stream, and
wherein the bypass stream comprises a carbon dioxide flow controller configured to control a flow rate of the contaminant feed stream into the Sabatier reactor by controlling flow rate of the bypass stream.

11. A method for generating methane, comprising:
absorbing, by a scrubber, one or more contaminants from an air stream into a liquid sorbent, wherein the one or more contaminants comprise carbon dioxide;
desorbing, by a stripper, the one or more contaminants from the liquid sorbent;
pressurizing, by a vacuum pump, the desorbed contaminants to a reaction pressure of the carbon dioxide;
removing, by a water separator, water from the pressurized, desorbed contaminants; and
generating, by a Sabatier reactor, the methane from the carbon dioxide of the pressurized, desorbed contaminants.

12. The method of claim 11, wherein the reaction pressure is less than 100 kilopascals (kPa).

13. The method of claim 11, wherein the vacuum pump is configured to draw a vacuum on the stripper.

14. The method of claim 11, further comprising heating, by a thermal link circuit using a heat transfer medium, the liquid sorbent with heat generated by the Sabatier reactor.

15. The method of claim 11, further comprising heating, by a heat exchanger using the methane from the Sabatier reactor, the liquid sorbent with heat generated by the Sabatier reactor.

16. The method of claim 11, further comprising:
receiving, by a first condenser, the methane from the Sabatier reactor;
receiving, by a second condenser, the carbon dioxide from the stripper;
receiving, by a second heat exchanger, the liquid sorbent from the stripper; and
removing, by a coolant loop, heat from the first condenser, the second condenser, and the heat exchanger in series.

17. The method of claim 11, further comprising generating, by a methane pyrolysis reactor, hydrogen from pyrolysis of the methane.

18. The method of claim 11, further generating, by an oxygen generation assembly, oxygen from electrolysis of generated water.

19. The method of claim 11, wherein generating the methane comprises controlling, by a control system, a rate of reaction of the Sabatier reactor based on at least one of a concentration of carbon dioxide in a contaminant stream discharged by the stripper or a flow rate of hydrogen entering the Sabatier reactor.

20. The method of claim 19,
wherein the pressurized, desorbed contaminants are split between a contaminant feed stream to the Sabatier reactor and a bypass stream, and
wherein controlling the rate of reaction of the Sabatier reactor comprises controlling a carbon dioxide flow controller to control a flow rate of the contaminant feed stream into the Sabatier reactor by controlling flow rate of the bypass stream.
